(19) United States
(12) Patent Application Publication
(10) Pub. No.: US 2009/0088791 A1
(43) Pub. Date: Apr. 2, 2009

(54) Carotid System Simplification

(75) Inventors: William J. Drasler, Minnetonka, MN (US); Mark L. Jenson, Greenfield, MN (US); Tracee Eidenschink, Wayzata, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 11/865,979

(22) Filed: Oct. 2, 2007

(51) Int. Cl. A61M 29/00; A61F 2/84

(52) U.S. Cl. 606/200; 623/1.11

(57) ABSTRACT

A method and apparatus for simplifying carotid artery stenting and/or angioplasty provides for the use of a filter wire system, which employs a sliding sheath. The sheath has an undeployed state and a deployed state, wherein in the undeployed state a distal region of the sheath is disposed about an embolic protection filter and a proximal region extends proximal from the distal region. At least a portion of the proximal region has a first end region, a second end region and a length there between. The first end region is proximal of the second end region. The at least a portion of the proximal region has a graduated stiffness along the length, wherein the stiffness is greatest at the first end region and is least at the second end region.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

In some embodiments this invention relates generally to methods, and systems for use in an interventional procedure of a stenosed or occluded region of a blood vessel. The systems and methods of the present invention are particularly useful when performing balloon angioplasty and/or, stenting procedures in critical vessels, where the release of embolic debris into the bloodstream could possibly occlude the flow of oxygenated blood to the brain or other vital organs. More specifically, some embodiments of the invention are directed to methods and systems for conducting Carotid Artery Stenting (CAS) and which provide significant improvements over known CAS methods and systems.

2. Description of the Related Art

Typical vascular disease involves the development of a stenosis in the vasculature. The particular vessel containing the stenosis can be completely blocked (or occluded) or it can simply be narrowed (or restricted). In either case, restriction of the vessel caused by the stenotic lesion results in many well known problems caused by the reduction or cessation of blood circulation through the restricted vessel.

Often, stenotic lesions are suitable for treatment by non-invasive techniques such as Percutaneous transluminal angioplasty (PTA), which involves advancement of a catheter equipped with a medical balloon to the lesion site, whereupon the balloon is expanded in order to increase blood flow through the affected vessel. In some cases a stent, or other endoprosthesis is implanted following and/or during the angioplasty procedure to reinforce the vessel and allow improved blood flow there through.

In some instances, a distal protection device, such as an embolic protection filter is inserted down stream of the lesion site in order to prevent emboli such as thrombi, plaque, and other embolic debris from drifting downstream and causing distal tissue injury. Most distal protection devices have filters that are attached directly to the distal portion of a guidewire or to a portion of a catheter. Filter devices can sometimes be used during surgery, during percutaneous interventional procedures, and also filters can be implanted permanently into the body. Some examples of filters are described in the following references: U.S. Pat. No. 5,910,154; U.S. Pat. No. 5,941,896; U.S. Pat. No. 5,928,261; U.S. Pat. No. 5,846,260; U.S. Pat. No. 5,810,874; U.S. Pat. No. 5,160,342; and U.S. Pat. No. 4,873,978 the entire contents of each being incorporated herein by reference.

Despite the significant benefits provided by using “non-invasive” treatments for the treatment of stenotic lesions, especially in the treatment of carotid artery disease, it is recognized that the advancement and manipulation of the various guidewires, catheters and other devices necessary to properly position the angioplasty balloon and/or stent delivery catheter can potentially lead to the dislodgement of embolic materials, such as thrombotic material and atherosclerotic plaque, which have the potential of being carried distally by the bloodstream into the cerebral vasculature and causing ischemic damage in the brain. This is of particular concern when the procedure involves a major vessel such as the carotid artery, such as during a CAS procedure. (See: Naylor et al, Randomized study of carotid angioplasty and stenting versus carotid endarterectomy: a stopped trial. J Vasc Surg 1998; 28:326 34; DeMonte et al, Carotid transluminal angioplasty with evidence of distal embolisation. J. Neurosurg 1989; 70:138 41; See also: Vitek J. J.; Technique of Carotid Angioplasty with Stenting. Russian Neurosurgery Online Journal (http://www.neuro.neva.ru/English/default.htm) 2000; Vol. 2.)

Given this recognized risk, filters, such as those described above are often used to reduce the chance of any freed emboli from passing beyond the filter and into the distal blood stream. Known non-invasive procedures, such as CAS, however do not deploy the filter until the procedure has already required several guidewire and/or catheter manipulations at or near (typically upstream) of the lesion site.

In PRIOR ART FIGS. 1-6, a stenotic area of the right interior carotid artery is depicted being treated in accordance with a known CAS method.

In PRIOR ART FIG. 1 a selective angiographic catheter (a.k.a.: diagnostic catheter) 10 is advanced to the ostium 20 of the right common carotid artery 22 along a standard 0.038 inch guidewire 12. The depicted anatomy is exemplary, because in many cases the target lesion 30 restricts flow into the internal carotid artery 24 and is often at or near the carotid bifurcation.

In PRIOR ART FIG. 2 the guidewire 12 is advanced into the external carotid artery 26 in order to provide subsequent system support to the advancement of a guide catheter 14, such as is illustrated in PRIOR ART FIG. 3.

In PRIOR ART FIG. 3 a catheter and/or a sheath such as an arterial sheath hereinafter identified collectively as guide catheter 14, is advanced into position in the ostium 20. In some procedures, such as in the example shown, the guide catheter 14 is advanced over the selective catheter 10. In many cases however, considerable manipulation and substitutions of the angiographic catheter 10 and/or other catheter(s) may be required in order to properly advance and position the guide catheter 14 as desired.

In PRIOR ART FIG. 4 the angiographic catheter 10 and the guidewire 12 are both withdrawn from the body, while the guide catheter 14 is left in place for subsequent use for the advancement of the filter wire 16 shown in PRIOR ART FIG. 5.

It must be noted, that as the aforementioned figures make abundantly clear, in the known CAS procedure depicted, there is no embolic protection mechanism in place during any of the stages described thus far or depicted in PRIOR ART FIGS. 1-4. Furthermore, known CAS procedures have no provision or mechanism for allowing the placement of an embolic protection filter, or similar device prior to these steps. It is only after all of this activity has occurred and after all of these various apparatuses have been inserted into the artery (angiographic catheter 10, guidewire 12, guide catheter 14, and filter wire 16), possibly multiple times, depending on the nature of the anatomy, that finally, do the known CAS procedures provide for the placement of some sort of embolic protection device.

As PRIOR ART FIG. 5 illustrates, a 0.014 inch filter wire 16 is, at last, passed through the guide catheter 14 and is advanced distally of the lesion 30 and into the internal carotid artery 24, whereupon an embolic protection filter 40 is deployed.

The last phase(s) of the known CAS procedure, is shown in PRIOR ART FIG. 6, wherein an angioplasty balloon catheter and/or a stent delivery system 18 is advanced along the filter wire 16 to dilate the lesion 30 and/or deliver a stent 19 across the lesion if necessary or desired. In some instances a subsequent angioplasty balloon catheter is used to post-dilate the lesion site if necessary or desired. After treatment the filter wire 16 is retrieved using a retrieval sheath (not shown) and the guide catheter 14 is withdrawn.

While it is certainly recognized that despite the absence of an embolic protection device distal of the lesion site during the initial phases of known CAS procedures the instance of embolism is believed to be remarkably small (see articles cited above), never the less, the risk does exist. Thus, there is a need in the art to provide for improved methods and apparatuses which further minimize the possibility of embolism during non-invasive procedures for the treatment of stenotic lesions, particularly in the carotid artery.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### BRIEF SUMMARY OF THE INVENTION

The present invention is directed in at least some embodiments to an apparatus and method for simplified CAS procedures through the use of a filter wire system, which avoids the necessity of an initial 0.038 inch guidewire, and which deploys an embolic protection device far earlier in the CAS process than current methods and/or systems.

In at least one embodiment the filter wire system employs an elongate wire, which has a diameter of about 0.010 of an inch to about 0.020 of an inch. In some embodiments the diameter of the wire is about 0.014 inch. About the wire is a sliding sheath, which has an undeployed state and a deployed state. In the undeployed state a distal region of the sheath is disposed about an embolic protection filter and a proximal region extends proximal from the distal region. At least a portion of the proximal region has a first end region, a second end region and a length there between. The first end region is proximal of the second end region. The at least a portion of the proximal region has a graduated stiffness along the length, wherein the stiffness is greatest at the first end region and is least at the second end region.

In some embodiments the sliding sheath is constructed out of a single material. In some embodiments the sliding sheath is constructed of different materials.

In some embodiments the sheath has a wall thickness, the thickness of the sheath wall at the proximal region tapers from a greatest thickness at the first end region to a least thickness at the second end region.

In some embodiments the sheath wall defines a plurality of grooves, cuts, notches, slits, etc, wherein the graduated stiffness is provided by the wall having a more and/or larger grooves at the second end region and fewer and/or smaller groves at the first end region. The groves can extend entirely or only partially through the sheath wall.

In at least one embodiment at least one grove extends substantially along the length of the proximal region according to a substantially helical or spiral pathway. The helical pathway extends about the circumference of the sheath wall in a plurality of complete circuits. The frequency of the circuits increases from the first end region to the second end region

In at least some embodiments at least one of the inner diameter and the outer diameter of at least the proximal region of the sheath wall is substantially constant along it length.

These and other aspects of the invention are described in more detail in the accompanying description and drawings.

### BRIEF DESCRIPTION OF THE SEVERAL VIEWS OF THE DRAWINGS

The invention is best understood from the following detailed description read in connection with the accompanying drawings.

PRIOR ART FIGS. 1-6 show a stylized cross-sectional view of an aorta and carotid tree of a potential human patient, wherein the internal branch of the right carotid artery is treated using a PRIOR ART CAS method and system.

FIG. 7 shows a longitudinal side view of an embodiment of the invention.

FIG. 8 shows an close-up, longitudinal, cross-sectional, perspective, view of the embodiment shown in FIG. 7, wherein the sheath is shown in the undeployed state.

FIG. 9 is a graphical illustration of the force deflection of the sheath component of the embodiment depicted in FIGS. 7-8.

FIG. 10 shows a longitudinal cross sectional view of an embodiment of the proximal region of the wire conversion sheath depicted in FIGS. 7-8.

FIG. 11 shows a longitudinal cross sectional view of an embodiment of the proximal region of the wire conversion sheath depicted in FIG. 7-8.

FIG. 12 shows a longitudinal cross sectional view of an embodiment of the proximal region of the wire conversion sheath depicted in FIG. 7-8.

FIG. 13 shows a longitudinal cross sectional view of an embodiment of the proximal region of the wire conversion sheath depicted in FIG. 7-8.

FIG. 14 shows a longitudinal perspective view of an embodiment of the proximal region of the wire conversion sheath depicted in FIG. 7-8.

FIG. 15-19 show a stylized cross-sectional view of an aorta and carotid tree of a potential human patient, wherein the internal branch of the right carotid artery is treated in accordance with an exemplary method and system embodied by the present invention.

### DETAILED DESCRIPTION OF THE INVENTION

The invention will next be illustrated with reference to the figures wherein the same numbers indicate similar elements in all figures. Such figures are intended to be illustrative rather than limiting and are included herewith to facilitate the explanation of the apparatus of the present invention.

For the purposes of this disclosure, like reference numerals in the figures shall refer to like features unless otherwise indicated.

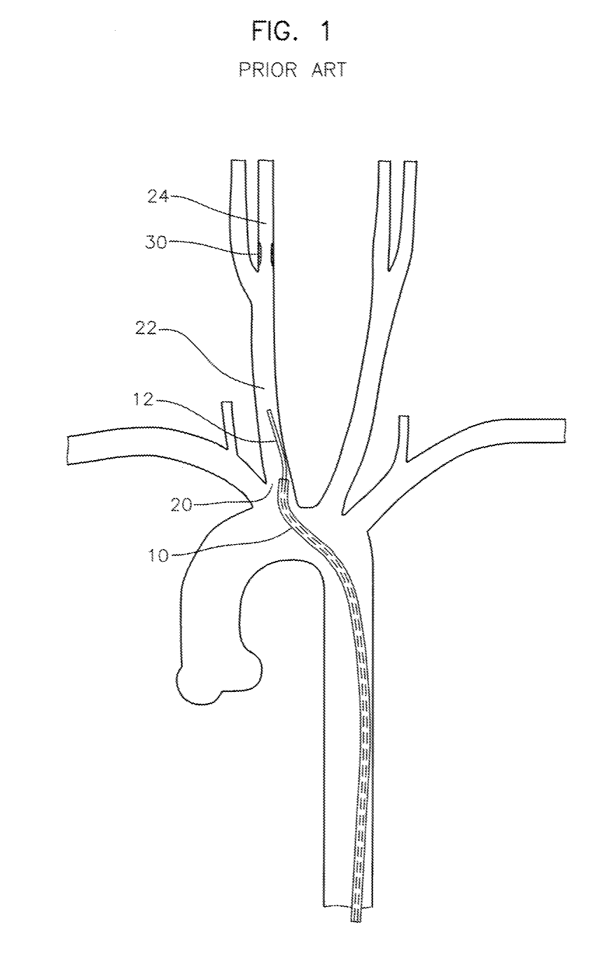

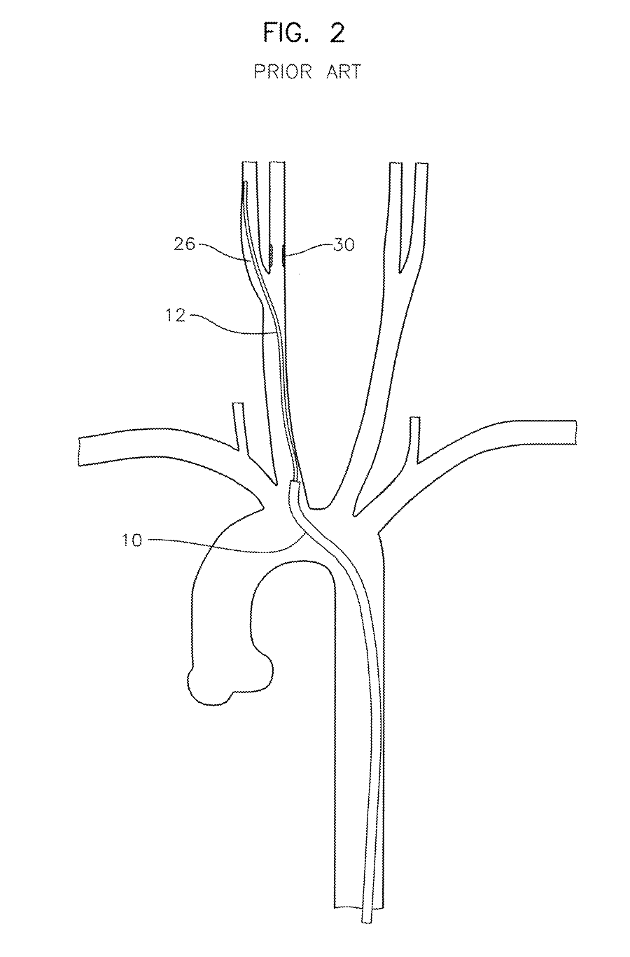

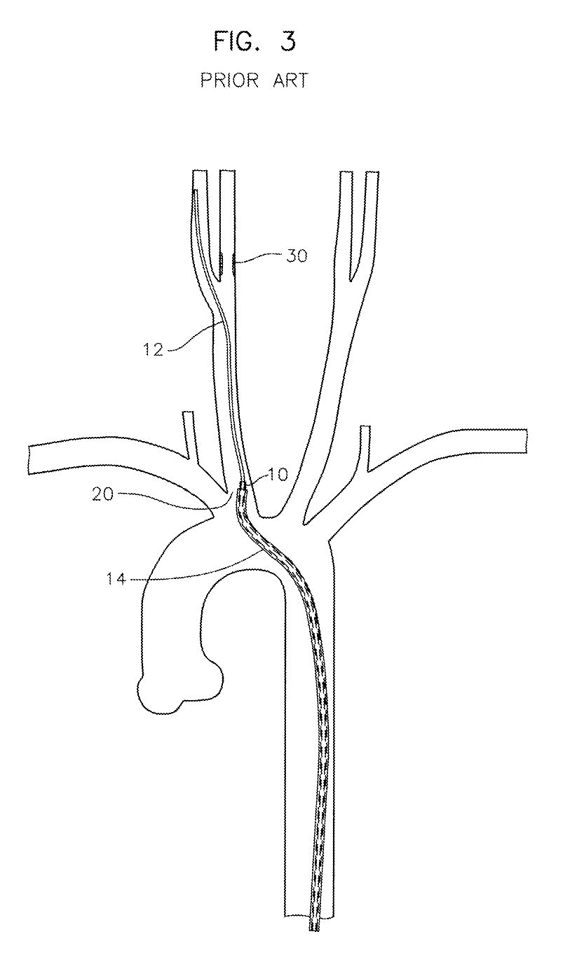

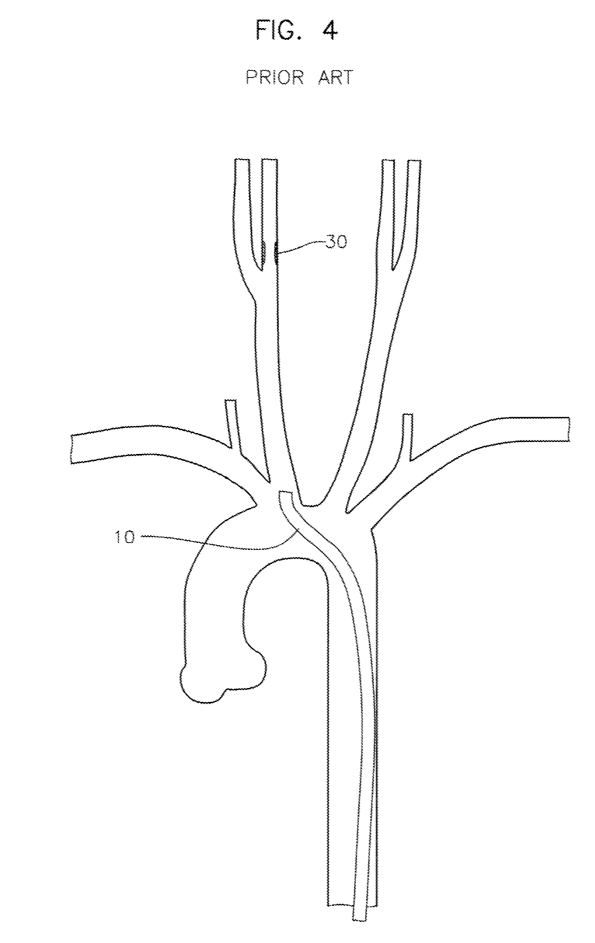

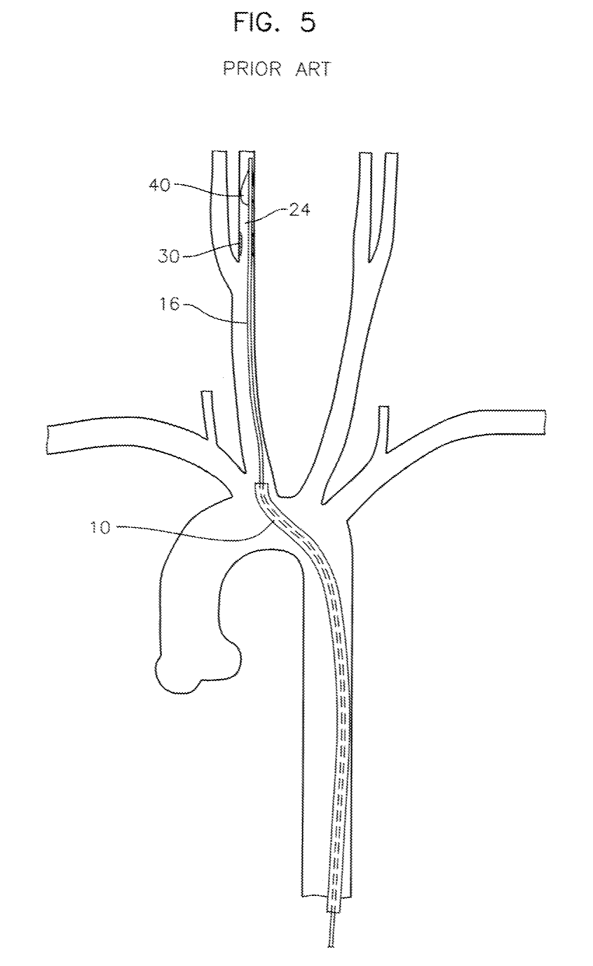

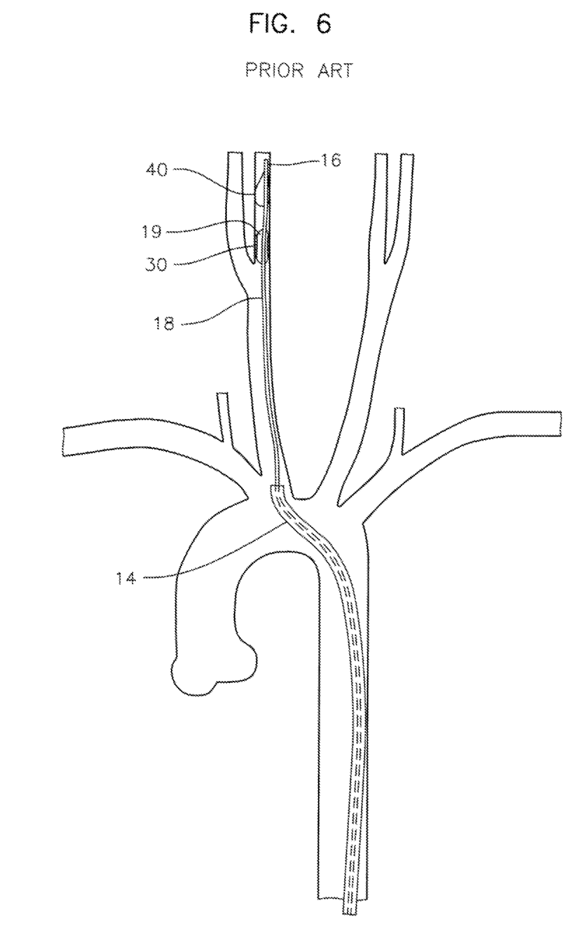

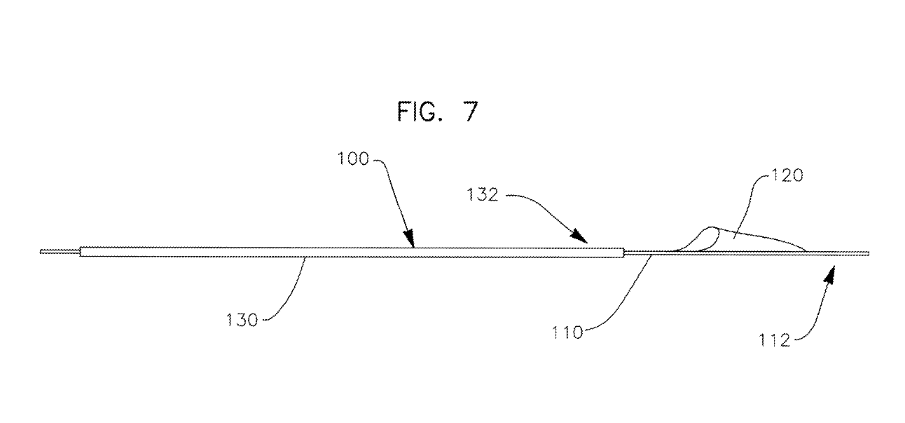

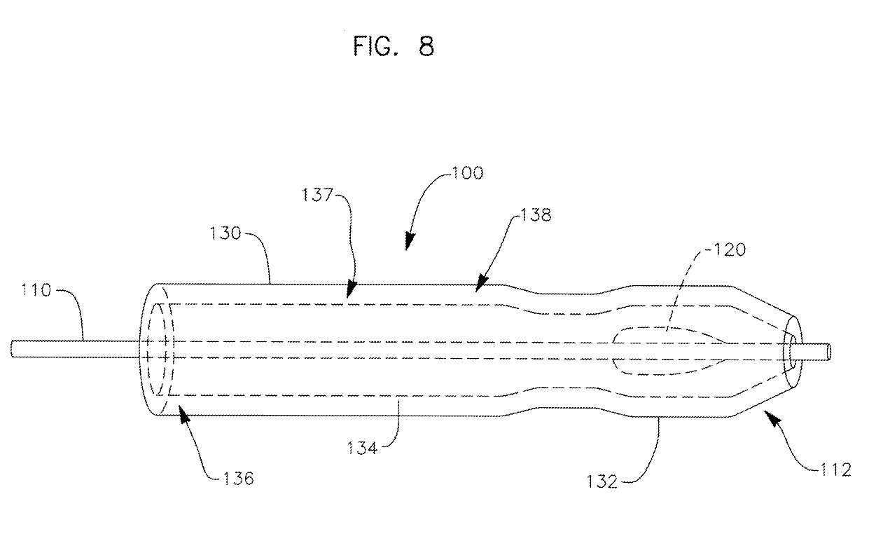

Referring now to FIGS. 7-8, there is shown a new filter wire system 100, which includes an elongate filter wire 110, equipped with an embolic protection filter 120 at or near its distal end 112. In at least one embodiment the elongate filter wire 110 of the present invention may be a standard 0.014 inch diameter wire. Other wire diameters suitable for use in the present invention may range from about 0.010 of an inch to about 0.020 of an inch.

The system 100 also includes a sliding sheath 130 disposed about the wire 110 and can slide along the length of the elongate wire 110 in order to act as a retention mechanism for the filter 120. FIG. 7 depicts the sheath 130 proximally withdrawn from the filter 120, to provide a deployed state wherein the filter 130 is free to deploy into the artery. In FIG. 8, the sheath 130 is shown prior to withdrawal from the filter 120, such that a distal region 132 of the sheath 130 remains positioned over the filter 120, thereby retaining the filter 120 in an undeployed state.

The majority of the length of the sheath 130 proximal to the distal (retaining) region 132 is referred to as the proximal region 134. This proximal region extends from a proximal end or first end region 136 of the wire, to the second end region 138, immediately adjacent to the distal retaining region 132. One or more sections of length between the first end region 136 and the second end region 138 can be characterized as a medial region 137.

A unique feature of the sheath 130 is that extending along the length of the proximal region 134, from the first end region 136, through the medial region(s) 137, to the second end region 138, the stiffness of the sheath 130 gradually decreases. This graduated stiffness provides at least a portion of the proximal region with a graduated force of deflection along its length. The relationship of the sheath's length relative to the force of deflection provided thereto, is illustrated in FIG. 9.

In some embodiments the sheath 130 may be characterized as having two, three, four or more distinct regions of differing stiffness. For example, in at least one embodiment the first end region 136 and the second end region 138 have distinct lengths with an established but different stiffness along those respective lengths. In at least one embodiment one or more medial regions 137 are located between the first end region 136 and the second end region 138. Each medial region likewise, may have a different stiffness than the regions adjacent thereto.

Another significant feature that the sheath 130 includes is an outer diameter 140, which ranges from as small as about 0.028 of an inch to no greater than about 0.040 of an inch. Some examples of specific diameters include: 0.030 inch, 0.032 inch, and 0.038 inch.

The combination of graduated stiffness along the length of the sheath 130, and especially along the length of the proximal region 134; with an outer diameter substantially equal to that of a guide wire (described above) allows the system 100 to be initially tracked through the vasculature in the same manner as a PRIOR ART guidewire 12 (shown in PRIOR ART FIGS. 1-3) and thus provide initial support and trackability to the subsequent procedure. At the same time however, the presence of the filter wire 110 and filter 120 within the sheath 130 allows the deployment of the filter 130 at the very initial stages of the CAS procedure, unlike the conventional method described above. Such initial use of the filter 120 is further described below and is depicted in FIGS. 15-19.

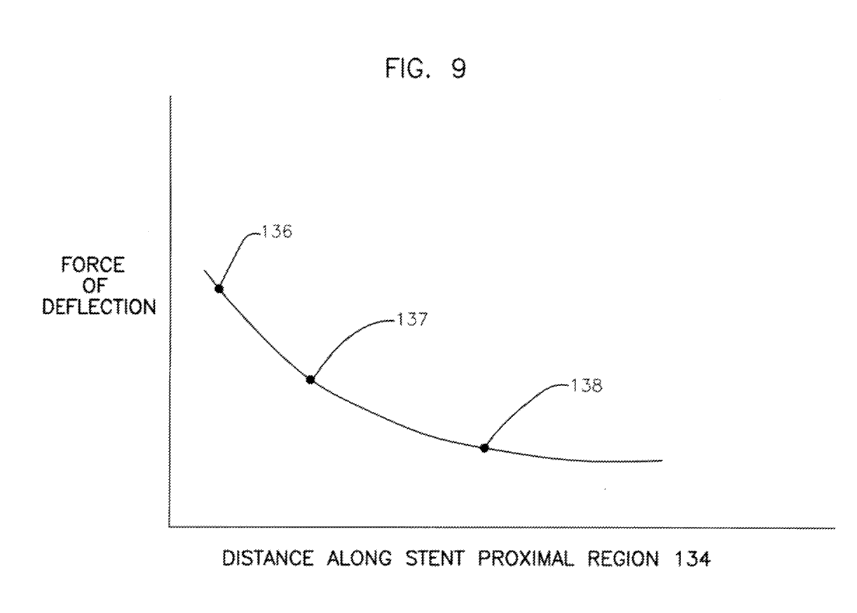

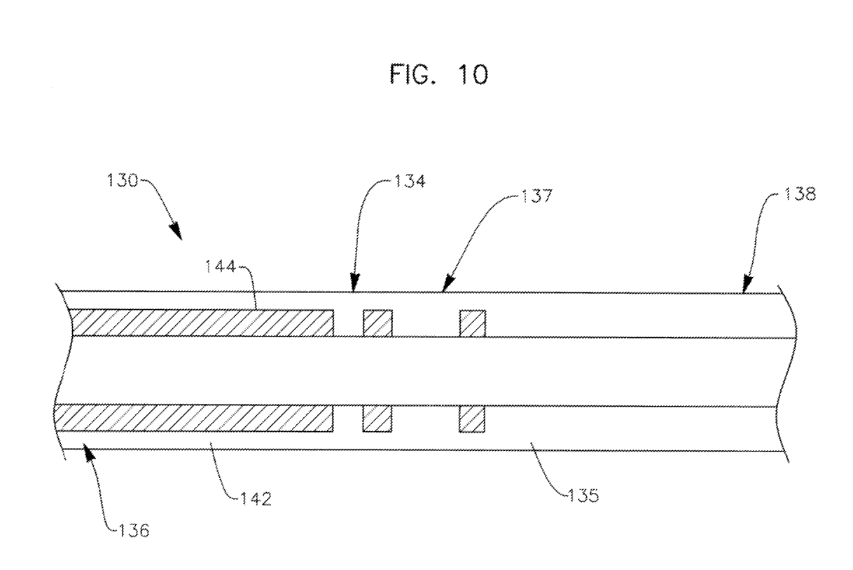

The unique graduated stiffness of the sheath 130 can be provided to at least the proximal region 134 of the sheath 130 in a variety of ways. In the embodiment shown in FIG. 10, for example, the sheath wall 135 is constructed from a plurality of different materials. In the embodiment shown, at least a portion of the first end region 136 is constructed from a combination of at least one first material 142 and at least one second material 144, wherein the at least one second material 144 has at least one material characteristic of being stiffer or harder than the at least one first material 142. The distribution of the at least one second material may be in the form of an additional layer or layers partially or entirely embedded or adjacent to the at least one first material. As shown in FIG. 10, in at least one embodiment the at least one second material 144, includes multiple discrete sections of material which are spaced along at least one medial region 137, to maintain the desired graduated stiffness of the sheath 130.

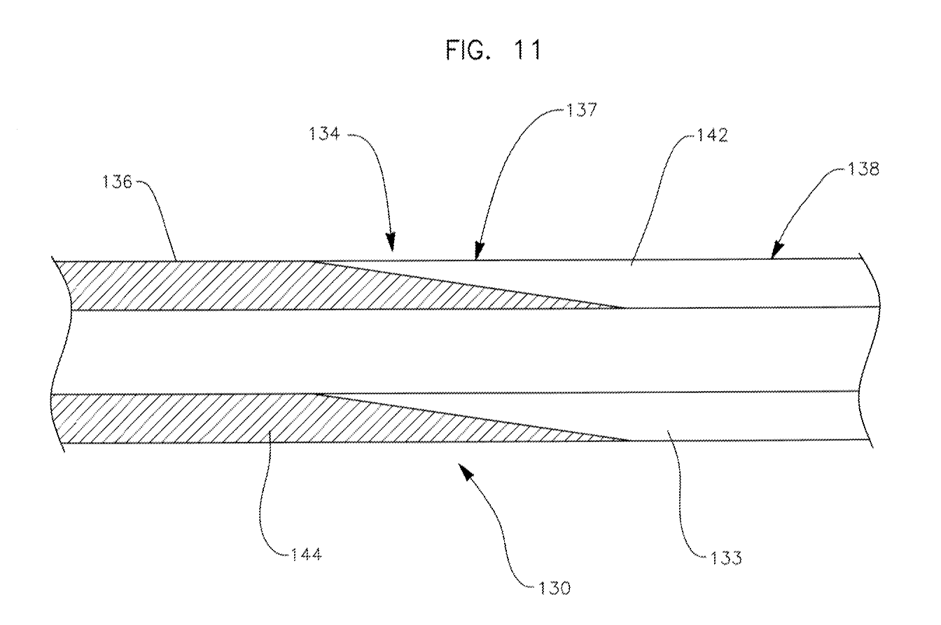

If desired the second end region 138 can in some embodiments, be constructed of entirely different material or materials than the first end region 136, with a medial region 137 providing a uniform transition between the differing materials 142 and 144 such as illustrated in FIG. 11.

Alternatively, in some embodiments the at least one second material can be distributed along the length of the sheath wall 135 in accordance with any of a variety of patterns (by co-extrusion, deposition, selective coating, etc.) to provide the first end region 136 with a greater concentration or distribution of the at least one second material 144 within or along the at least one first material 142, compared to a reduced concentration or distribution of the at least one second material 144 at the second end region 138.

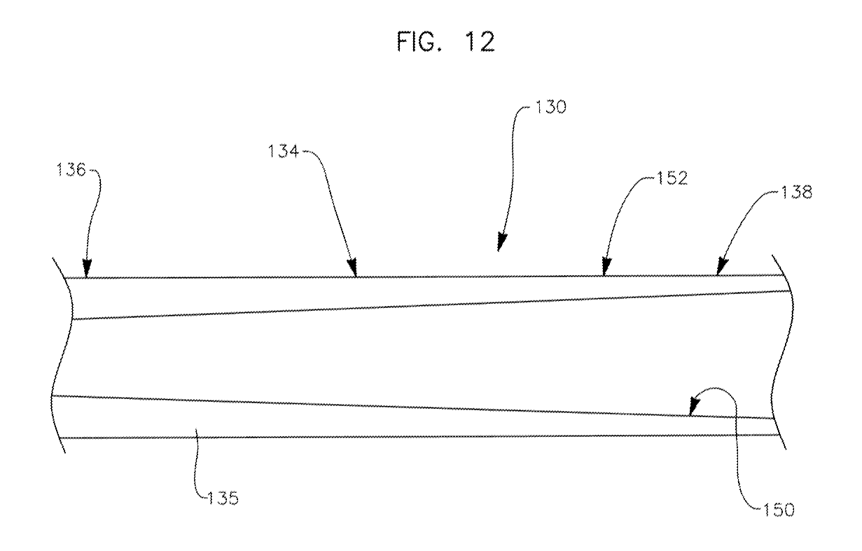

In some embodiments, the graduated stiffness of the sheath 130 is provided by forming the sheath wall 135 to include a tapered thickness along the length of at least a portion of the proximal region 134, such as in the manner depicted in FIG. 12. The taper would provide for the greatest wall thickness at the first end region 136 and the thinnest wall thickness at the second end region 138. The taper of the wall thickness can result in a tapered inner diameter 150 or a tapered outer diameter 152 as desired. In the embodiment shown in FIG. 12, the outer diameter 152 of the sheath is maintained at no greater than about 0.038 of an inch along its entire length. In an embodiment such as is depicted in FIG. 12, the tapered inner diameter 150 of the proximal region of the sheath can be extended distally beyond the proximal region 134 and into at least a portion of the distal region 132, in order to provide additional lumen space for the filter 120 if desired or necessary.

Sheath 130 can be constructed from a variety of materials including polymeric and/or metallic compositions. In the various embodiments described herein the material of the sheath and/or the filter wire lumen which the sheath defines includes a lubricious material and/or coating to minimize resistance between the filter wire and the sheath. In some embodiments the lubricious nature of the filter wire lumen is an inherent property of the material from which the sheath wall 135 is constructed. In some embodiments, the filter wire is provided with a lubricous coating.

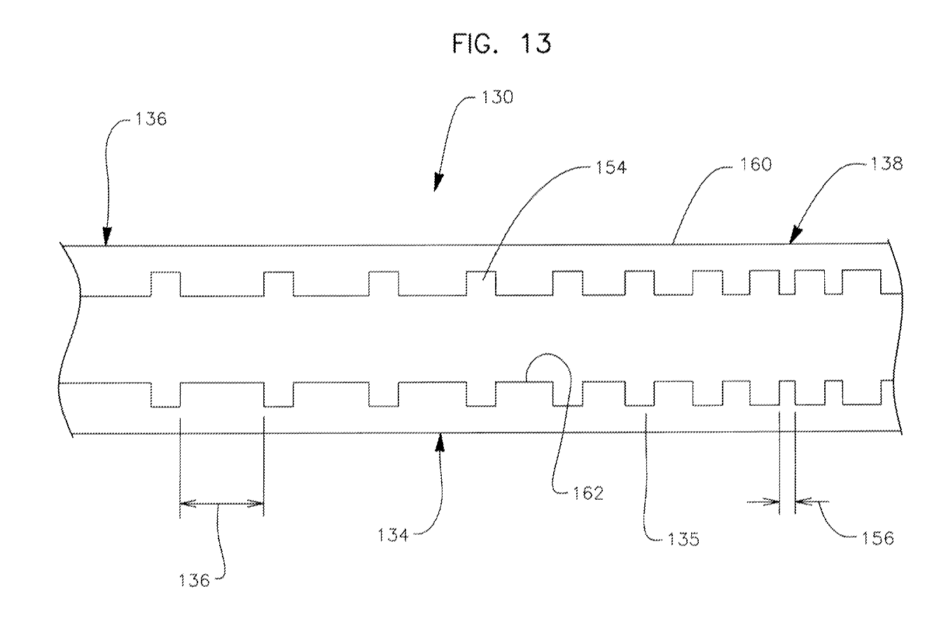

In at least one embodiment, an example of which is shown in FIG. 13, the sheath wall 135 is constructed of a slotted tube of nitinol. As indicated the wall 135 defines a plurality of slots (a.k.a. notches, grooves, openings, holes, etc) 154. Each of the slots 154 have a depth which extends through a portion of the wall thickness. The slots 154 are spaced apart from one another by a slot distance 156. The slot distance 156 decreases from a greatest slot distance between the slots in the first end region 136 of the to a lesser slot distance 156 between the slots 154 in the second end region 138.

In some embodiments the number of slots 154 per a given unit of length of the proximal region 134 increases from the first end region 136 to the second end region 138.

In some embodiments, the depth of the slots may also be increased on a gradual basis extending from the first end region 136 to the second end region 138.

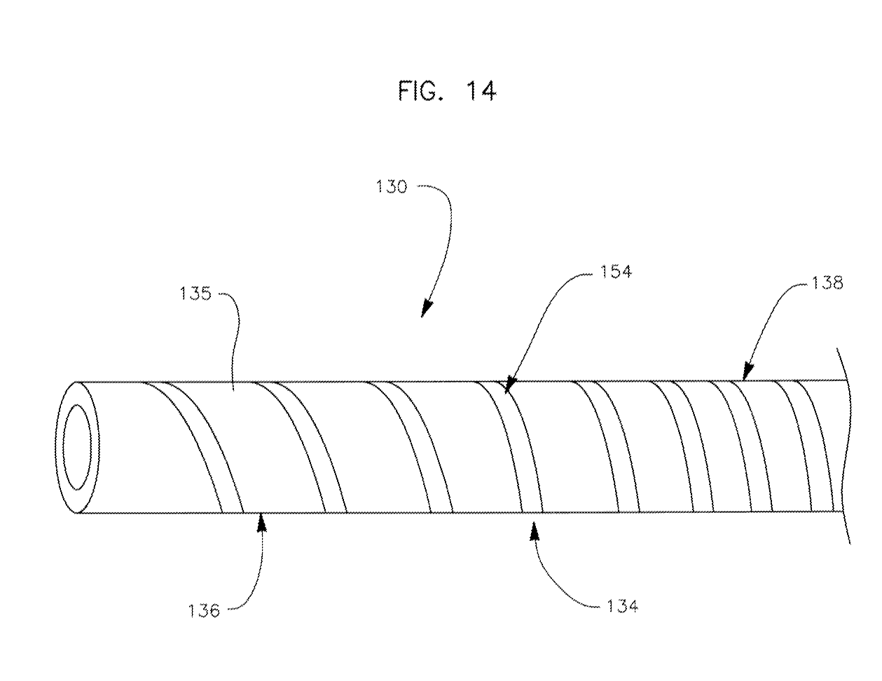

In at least one embodiment, an example of which is shown in FIG. 14 the sheath wall 135 defines a single helical or spiral slot or groove 154 which extends, uninterrupted, from at least a portion of the first end region 136 to at least a portion of the second end region 138 about the circumference of the sheath 130. As the helical grove 154 extends along the length of at least the proximal region 134, the groove 154 will repeatedly circumnavigate the sheath wall 135. The frequency of complete circumnavigations or circuits will increase from the first end region 136 to the second end region 138 in the manner depicted.

In various embodiments, slots or grooves 154 may extend entirely through the thickness of the sheath wall 135 or may merely extend to a predetermined depth therein. Such ‘closed bottom’ slots or grooves 154 may be open at either the outer surface 160 of the sheath wall or the inner surface 162 of the sheath wall as desired. Construction of such slotted sheaths can be provided for according to a variety of techniques including but not limited to: using a textured mandrel upon which the sheath is formed to provide ‘internal’ slots; coating or depositing material on the external surface of a sheath blank to provide ‘external’ slots; laser, mechanical, chemical etching to selectively remove material from the sheath blank to form slots, etc.

Regardless of the manner in which graduated stiffness is imparted to the sheath 130, the unique characteristics of the sheath 130 when combined with the filter wire 110 and filter 120 provide for a system that is advanced to a lesion site to provide embolic protection, much earlier in the treatment procedure, when compared to a conventional CAS procedure, such as that previously shown and described.

In the FIGS. 15-19 an embodiment of the filter wire system 100 described above is depicted being utilized in a new and simplified CAS procedure now made possible by the innovations described above.

In FIG. 15 the system 100 is advanced into the right common carotid artery 22. If the position of the lesion 30 is known, then the distal region 132 of the system can be advanced downstream of the lesion site, whereupon the sheath 130 is withdrawn from the undeployed position to the deployed position to allow with filter 120 to expand. If however, the precise position of the lesion is yet unknown, an angiographic catheter 10 can be advanced over the system 100.

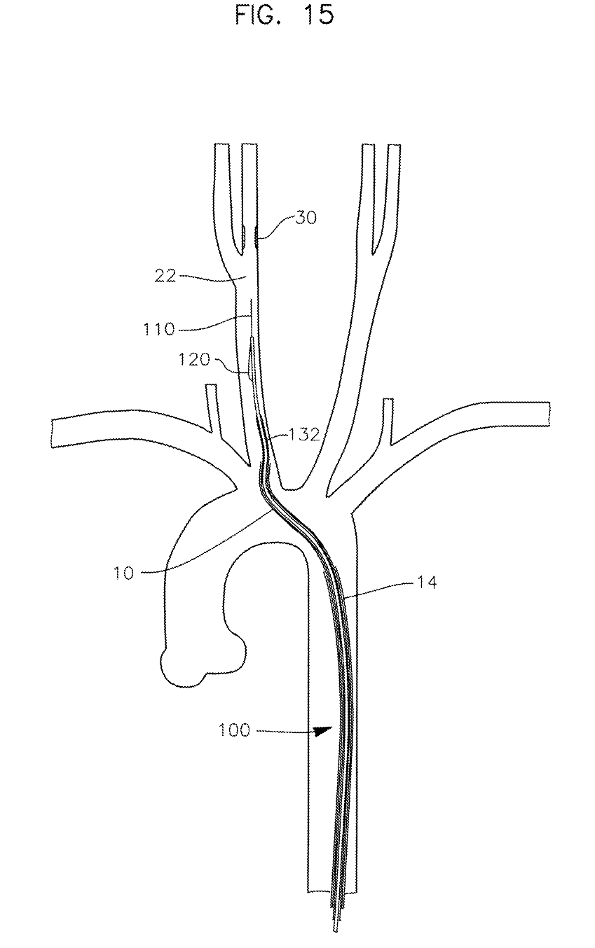

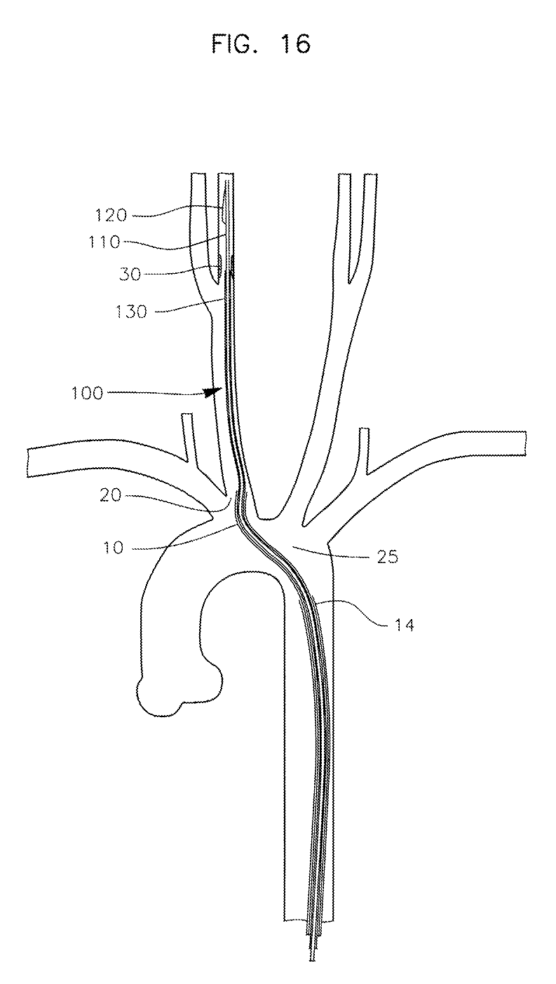

As shown in FIG. 16, a guide catheter 14 can be advanced over the system 100 and/or the angiographic catheter 10 prior to or subsequent to deployment of the filter 120. If the guide catheter 14 is advanced prior to the deployment of the filter 120, the guide catheter 14 remains in the aorta 25 until the filter 120 is properly positioned and deployed distal of the lesion 30.

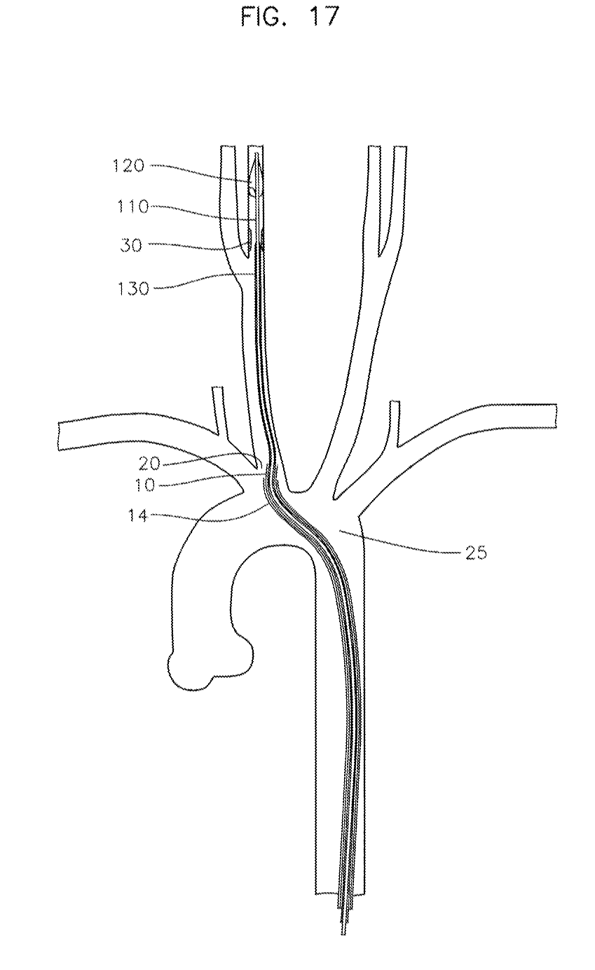

Once the filter 120 is deployed, the filter wire 110 and sheath 130 provide sufficient support to allow the guide catheter 14 to be advanced into the ostium 20 in the manner depicted in FIG. 17. During this advancement the filter 120 provides embolic protection during the manipulations of not only the guide catheter 14, but all other subsequent CAS steps.

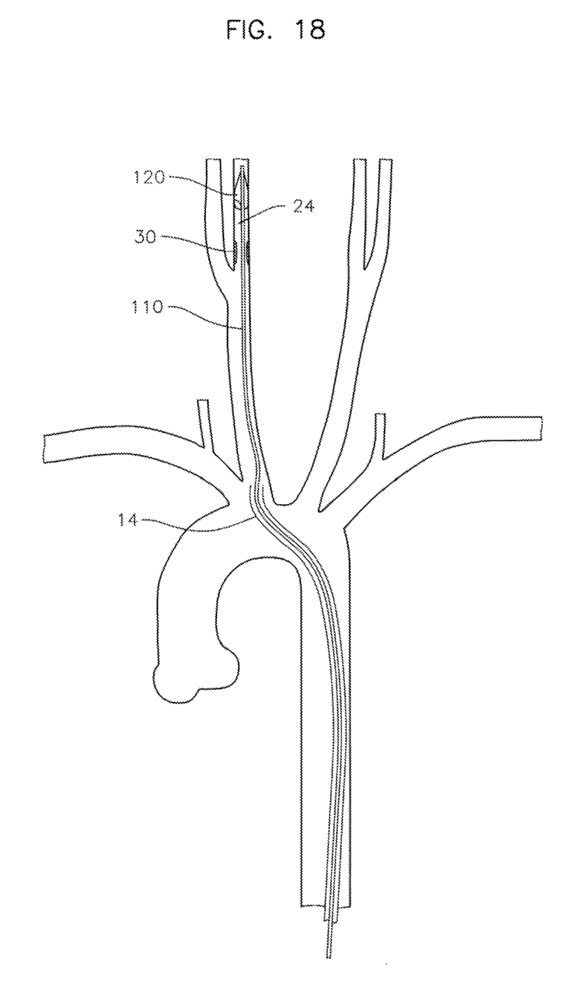

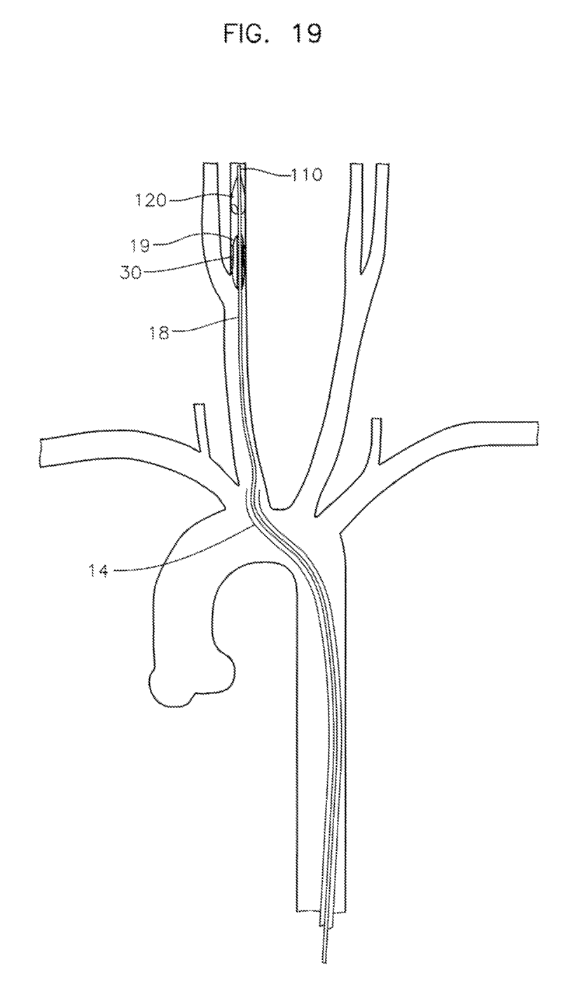

Next, as depicted in FIG. 18 the simplified CAS procedure, allows for withdrawal of the angiographic catheter 10 (if it has not yet been removed) as well as the sheath 130, leaving the filter wire 110 (and the deployed filter 120) and guide catheter 14 in place to accommodate subsequent advancement and use of one or more angioplasty balloon catheter(s) and/or stent delivery catheter(s)/system(s) 18, such as shown in FIG. 19.

A stent delivery system 18 can be advanced along the filter wire 110 to the lesion site, whereupon the stent 19 is deployed across the lesion 30 in the manner shown.

Following stent deployment, an angioplasty balloon can be used to post-dilate the stent. After treatment is complete the filter wire 110 and filter 120 is retrieved using a retrieval sheath (not shown) and the guide catheter 14 is withdrawn.

It should be recognized from the above description, particularly when the conventional CAS method described and shown in FIGS. 1-6 is compared to the improved and simplified method described and shown in FIGS. 15-19 that the present invention provides a significant improvement in embolic protection over the conventional method. Moreover, the graduated stiffness and reduced profile of the filter wire system 100 provides an additional increase in the efficiency of a CAS procedure by eliminating several steps of the procedure as the aforementioned comparison makes clear.

This completes the description of the preferred and alternate embodiments of the invention. The above disclosure is intended to be illustrative and not exhaustive. This description will suggest many variations and alternatives to one of ordinary skill in this art. The various elements shown in the individual figures and described above may be combined, substituted, or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claims below.

1. A filter wire system comprising:
an elongate wire, the elongate wire having a diameter of about 0.010 of an inch to about 0.020 of an inch;
an embolic protection filter, the embolic protection filter being positioned at a distal end region of the elongate wire; and
a sliding sheath, the sheath having an undeployed state and a deployed state, in the undeployed state a distal region of the sheath is disposed about the embolic protection filter and a proximal region extends proximal from the distal region, at least a portion of the proximal region having a first end region, a second end region and a length there between, the first end region being proximal of the second end region, the at least a portion of the proximal region having a graduated stiffness along the length, wherein the stiffness is greatest at the first end region and least at the second end region.

2. The system of claim 1 wherein the elongate wire has a diameter of about 0.014 of an inch.

3. The system of claim 1 wherein the sheath has an outer diameter of about 0.028 of an inch to about 0.040 of an inch.

4. The system of claim 1 wherein the sheath has an outer diameter no greater than about 0.038 of an inch.

5. The system of claim 1 wherein the sheath is al least partially constructed of a lubricious polymer material.

6. The system of claim 1 wherein the sheath comprises a sheath wall, the sheath wall is constructed of a plurality of materials, at least a portion of the first end region comprising at least one first material and at least one second material, at least a portion of the second end region consisting of only the at least one first material.

7. The system of claim 1 wherein the sheath provides the at least a portion of the proximal region with a graduated force of deflection along the length, wherein the force of deflection is greatest at the first end region and least at the second end region.

8. The system of claim 1 wherein the sheath comprises a sheath wall, the sheath wall having a thickness, the thickness of the sheath wall of the at least a portion of the proximal region tapering from a greatest thickness at the first end region to a least thickness at the second end region.

9. The system of claim 1 wherein the sheath comprises a sheath wall, the sheath wall having a thickness, the sheath wall defining at least one slot, the at least one slot having a depth which extends through a portion of the thickness, the at least one slot extending helically about the sheath wall in a plurality of complete circumferential circuits, the frequency of circuits increasing from the first end region of the at least a portion of the proximal region to the second end region.

10. The system of claim 9 wherein the sheath wall has an inner diameter and an outer diameter, at least one of the inner diameter and the outer diameter being substantially constant along the length of the at least a portion of the proximal region.

11. The system of claim 1 wherein the sheath comprises a sheath wall, the sheath wall having a thickness, the sheath wall defining a plurality of slots, each of the slots having a depth which extends through at least a portion of the thickness, the slots being spaced apart from one another by a slot distance, the slot distance decreasing from a greatest slot distance between the slots in the first end region of the at least a portion of the proximal region to a smallest slot distance between the groves in the second end region.

12. The system of claim 11 wherein plurality of slots increases from the first end region of the at least a portion of the proximal region to the second end region.

13. The system of claim 12 wherein the sheath wall has an inner diameter and an outer diameter, at least one of the inner diameter and the outer diameter being substantially constant along the length of the at least a portion of the proximal region.

14. The system of claim 1 wherein the at least a portion of the proximal region having a first end region, a second end region and a medial region there between, the first end region being proximal of the second end region, the at least a portion of the proximal region having a graduated stiffness along the length, wherein the stiffness of the first end region is greater than that of the medial region, and the stiffness of the medial region is greater than that of the second end region.

15. A method for stenting a carotid artery without the use of a guide wire comprising:
advancing a filter wire system into the carotid artery, the filter wire system comprising:
an elongate wire, the elongate wire having a diameter of about 0.010 of an inch to about 0.020 of an inch,
an embolic protection filter, the embolic protection filter being positioned at a distal end region of the elongate wire,
a sliding sheath, the sheath having an undeployed state and a deployed state, in the undeployed state a distal region of the sheath is disposed about the embolic protection filter and a proximal region extends proximal from the distal region,
at least a portion of the proximal region having a first end region, a second end region and a length there between, the first end region being proximal of the second end region, the at least a portion of the proximal region having a graduated stiffness along the length, wherein the stiffness is greatest at the first end region and least at the second end region;
deploying the embolic protection filter at a location distal of a lesion site in the carotid artery;
advancing a guide catheter over the filter wire system to a location proximal of the lesion site;
removing the sliding sheath from the carotid artery;
advancing a stent delivery system over the elongate wire, and within the guide catheter to the lesion site; and
deploying a stent across the lesion site.

16. The method of claim 15 further comprising:
advancing a diagnostic catheter to a position proximal of the lesion site in the carotid artery.

17. The method of claim 16 further comprising:
removing the diagnostic catheter from the carotid artery before advancing the stent delivery system over the filter wire.
